(19) United States
(12) Patent Application Publication
(10) Pub. No.: US 2020/0368383 A1
(43) Pub. Date: Nov. 26, 2020

(54) METHOD FOR FORMING CHANGEABLE MIST OF AROMATHERAPY MACHINE AND APPLIED AROMATHERAPY MACHINE

(71) Applicant: Foshan Nanhai Keri Electronic Co.,Ltd, Guangdong (CN)

(72) Inventors: Jinyun FENG, Guangdong (CN); Linfu SHEN, Guangdong (CN); Guoliang CHEN, Guangdong (CN)

(73) Assignee: Foshan Nanhai Keri Electronic Co.,Ltd, Guangdong (CN)

(21) Appl. No.: 16/959,435

(22) PCT Filed: Jul. 22, 2019
(86) PCT No.: PCT/CN2019/097082

§ 371 Date: Jul. 1, 2020

(30) Foreign Application Priority Data
Dec. 21, 2018 (CN) 201811571788.6
Dec. 21, 2018 (CN) 201822163527.2
Dec. 21, 2018 (CN) 201822163566.2
Apr. 12, 2019 (CN) 201910292743.3

(51) Int. Cl. A61L 9/14; A61M 21/00; A61M 11/00

(52) U.S. Cl. CPC A61L 9/14; A61M 21/00; A61M 11/005; F24F 6/12; A61M 2021/0016; A61M 2205/3331; A61M 2205/584; A61L 2209/134

(57) ABSTRACT

A method for forming a changeable mist of an aromatherapy machine and the aromatherapy machine are provided. A mist storage cavity with an upward opening is disposed on the aromatherapy machine, and a mist outlet of the aromatherapy machine communicates with an inner space of the mist storage cavity. When the aromatherapy machine generates an atomized vapor in an atomization chamber, the atomization chamber is provided with an internal air pressure higher than an atmospheric pressure, so that the atomized vapor is ejected outward at a different speed state based on a different internal air pressure, so as to form a different mist retention state. In a first configuration solution, the internal air pressure is controlled to be slightly higher than an external atmospheric pressure, so that the atomized vapor floats out in a slow surge manner, and that the atomized vapor is accumulated as a haze in the inner space.

### BACKGROUND

### Technical Field

The invention relates to a method for forming a changeable mist of an aromatherapy machine and an applied aromatherapy machine. The aromatherapy machine can not only adjust the aroma or humidity of an indoor environment, but also form an effect of a changeable mist, which has a certain degree of ornamental value.

### Description of Related Art

The aromatherapy machine is essentially a humidifier, and is generally equipped with an ultrasonic atomizing sheet and a blower. The basic principle of generating cavitation bubbles using the ultrasonic high-frequency oscillation of the ultrasonic atomizing sheet is to break up atomized liquid such as water and aroma liquid, thereby generating mist that can drift. The resulting mist is ejected from an atomizing hole by the strong air flow from the blower and floated into the high air. When the aromatherapy machine is used in different application spaces, in order to specifically provide the surrounding environment with a suitable humidity, it is necessary to configure different spray amounts. Therefore, some of the aromatherapy machines are often equipped with a mist output amount adjusting device. For example, a large ultrasonic humidifier with adjustable atomization amount described in Chinese utility model CN201420093742.9 belongs to the field of large humidifiers, including a casing and a fan. An atomization chamber is provided in the casing. The atomization chamber is provided with an air inlet, an air outlet, and an atomizer. The atomizer is provided in the atomization chamber. The fan sends air to the atomization chamber via an air inlet. A humidity detector, a humidity controller, a silicon controlled rectifier, and a transformer connected with the silicon controlled rectifier are also included. The humidity detector detects the ambient humidity and generates a humidity signal to the humidity controller. The humidity controller converts the humidity signal into a control signal and sends it to the silicon controlled rectifier. The silicon controlled rectifier converts the control signal into an adjustment signal and sends it to the atomizer. The atomizer adjusts the amount of atomization according to the adjustment signal. The large ultrasonic humidifier can keep the humidity of the use environment constant, avoiding the phenomenon of larger fluctuations in the humidification effect.

The technical solution represented by the above patent proposes how to adjust the amount of atomization to improve the indoor humidity and aroma density. However, currently consumers who purchase and use aromatherapy machines in their daily lives pay special attention to the sensibility of life and the coordination of daily necessities, environment, and their mood. When consumers want to use the aromatherapy machine to express their mood or use the aromatherapy machine to improve the joy of life, it is obviously not enough to adjust the amount of atomization of the aromatherapy machine.

### SUMMARY

In view of the above technical issues existing in the prior art, in order to further improve the life experience of the aromatherapy machine, especially to reflect and express a user's mood and increase the ornamental value and pleasant feeling of the aromatherapy machine, the invention provides a method for forming a changeable mist of an aromatherapy machine. Providing a mist storage cavity with an upward opening on the aromatherapy machine, wherein a mist outlet of the aromatherapy machine communicates with an inner space of the mist storage cavity. When the aromatherapy machine generates an atomized vapor in an atomization chamber, providing the atomization chamber with an internal air pressure higher than an atmospheric pressure, so that the atomized vapor may be ejected outward from the mist outlet at a corresponding different speed state based on a different internal air pressure to form a different mist retention state on an outside of the aromatherapy machine. Providing a plurality of internal air pressure configuration solutions, wherein in a first configuration solution, the internal air pressure of the atomization chamber is controlled to be slightly higher than an external atmospheric pressure, so that the atomized vapor floats out in a slow surge manner, and that the atomized vapor is mainly accumulated as a haze in the inner space of the mist storage cavity, and in a second configuration solution, the internal air pressure of the atomization chamber is controlled to be higher than the internal air pressure controlled in the first configuration solution, so that the atomized vapor is ejected from the mist outlet in a fast impulse manner, and that the atomized vapor is mainly scattered in a space above the mist storage cavity.

In particular, the plurality of internal air pressure configuration solutions refer to that the first configuration solution and the second configuration solution may be merely provided, or other configuration solutions are also provided in addition to the first configuration solution and the second configuration solution.

In particular, the atomized vapor is composed of countless tiny water droplets. Under natural conditions, the atomized vapor naturally sinks under the action of gravity. Therefore, the atomized vapor obtains the ejection kinetic energy via the air pressure difference between the internal air pressure of the atomization chamber of the aromatherapy machine and the atmospheric pressure. The greater the internal air pressure of the atomization chamber, the greater the ejection kinetic energy of the atomized vapor can be obtained, and the higher the injection speed, and the diffusion speed in the air after ejection is different. Therefore, different internal air pressures of the atomization chamber cause the atomized vapor to have different ejection kinetic energy and diffusion kinetic energy, thereby forming different mist retention states outside the aromatherapy machine. In the first configuration solution, the internal air pressure of the atomization chamber is slightly higher than the external atmospheric pressure, the kinetic energy obtained by the atomized vapor in the atomization chamber is relatively smaller, and the atomized vapor floats out of the mist outlet in a slow surge manner. Most of the atomized vapor sinks under the effect of its own weight and gradually accumulates in the inner space of the mist storage cavity to form a haze-like mist retention state, forming a mountainous thick mist that slowly rises and is diffused from the periphery of the mist storage cavity and slowly drifted downward as if a plume of fine smoke drifting from up high. In the second configuration solution, the internal air pressure of the atomization chamber is higher than the internal air pressure controlled by the first configuration solution, the kinetic energy obtained by the atomized vapor in the atomization chamber is relatively larger, and the atomized vapor is ejected from the mist outlet in a fast impulse manner. Most of the atomized vapor does not stay temporarily in the inner space of the mist storage cavity but continues to be ejected upward and then away from the mist storage cavity into the space above the mist storage cavity and is gradually diffused to form a mist retention state drifting around. A mist output scene as if mist was ejected high from a fountain is formed, and a small portion of atomized vapor or even no atomized vapor sinks into the mist storage cavity.

To enrich the mist output effect, another technical solution may be that the mist storage cavity is a light-transmitting body, and a changing light is disposed below the mist storage cavity to set off the atomized vapor. In this way, the atomized vapor generated by the aromatherapy machine can be stored to be colorful under the changing lights for a better viewing effect.

Another technical solution may be that the internal air pressure of the atomization chamber is continuously and variably controllable. In this way, a plurality of internal air pressures can be formed, so as to form a plurality of different mist output scenes.

There are a plurality of methods for adjusting the internal air pressure of the atomization chamber, and one of the pressure adjusting methods is further provided below, the method further including an air channel providing an air supply amount to the atomization chamber, and controlling the air supply amount provided to the atomization chamber via the air channel to configure the internal air pressure of the atomization chamber. On this basis, the following air amount adjusting methods can be further adopted.

A first adjusting method in which a size of an air outlet is changed via an air valve disposed on the air channel to control the air supply amount provided to the atomization chamber via the air channel.

A second adjusting method in which the air supply amount provided to the atomization chamber via the air channel is controlled by controlling a rotating speed of a blower communicating with the air channel.

In addition, the invention further provides an aromatherapy machine implementing the method for forming a changeable mist of the aromatherapy machine. The aromatherapy machine includes an atomization chamber disposed in a body of the aromatherapy machine, and further includes a mist outlet arranged in an upward direction. The mist outlet communicates with the atomization chamber to discharge an atomized vapor in the atomization chamber to a space outside the machine in the upward direction. A mist storage cavity with an opening upward is disposed on the aromatherapy machine, and the mist outlet of the aromatherapy machine is communicated with an inner space of the mist storage cavity. The aromatherapy machine further includes an adjusting device, wherein the adjusting device is configured to adjust an internal air pressure of the atomization chamber in a plurality of pressure configuration solutions, so that the atomized vapor may be ejected outward from the mist outlet at a corresponding different speed state based on a different internal air pressure to form a different mist retention state on an outside of the aromatherapy machine. In a first configuration solution, the adjusting device is configured to control the internal air pressure of the atomization chamber is controlled to be slightly higher than an external atmospheric pressure, so that the atomized vapor floats out in a slow surge manner, and that the atomized vapor is mainly accumulated as a haze in the inner space of the mist storage cavity. In a second configuration solution, the adjusting device is configured to control the internal air pressure of the atomization chamber to be higher than the internal air pressure controlled in the first configuration solution, so that the atomized vapor is ejected from the mist outlet in a fast impulse manner, and that the atomized vapor is mainly scattered in a space above the mist storage cavity.

Another technical solution may be that the mist storage cavity is a light-transmitting body, a light-emitting body assembly that has changeable colors is disposed below the mist storage cavity, and the light-emitting body assembly is configured to set off the atomized vapor with a light emitted thereby.

Another technical solution may be that the mist storage cavity is integrally formed as one piece with a top portion of the aromatherapy machine, or the mist storage cavity is independent from the top portion of the aromatherapy machine and the mist storage cavity is detachably installed on the top portion of the aromatherapy machine.

Another technical solution may be that an air channel communicating with the atomization chamber is further included, wherein the air channel is configured to provide an air supply amount to the atomization chamber; the adjusting device is configured to control the air supply amount provided to the atomization chamber via the air channel to adjust the internal air pressure of the atomization chamber.

The adjusting device may further adopt the following two structures.

A first adjusting structure, wherein the adjusting device is an air valve disposed on the air channel, and the air valve is configured to control the air supply amount provided to the atomization chamber via the air channel by changing a size of the air outlet. In addition, a drive motor mounted on a body of the aromatherapy machine is further included, an output shaft of the drive motor is drivingly connected to the air valve, and the drive motor is configured to drive an action of the air valve to change the size of the air outlet of the air valve.

A second adjusting structure further includes a blower communicating with the air channel, wherein the adjusting device is an electronic control device disposed on the body of the aromatherapy machine to control a rotating speed of the blower, and the electronic control device is configured to change the rotating speed of the blower to control the air supply amount provided to the atomization chamber via the air channel.

Another technical solution may be that a first blower and a second blower respectively communicating with the air channel are further included, wherein an air supply amount of the first blower is greater than an air supply amount of the second blower, the adjusting device is an electronic control device disposed on the body of the aromatherapy machine to control the first blower and the second blower to be operated alternately, and an operating state of the first blower or the second blower is changed via the electronic control device to control the air supply amount provided to the atomization chamber via the air channel. In particular, alternate operation means that when the first blower is in operation, the second blower stops operating. Or, when the second blower is in operation, the first blower stops operating. In this way, the first blower can provide a relatively large air volume to the air channel during operation, and can provide a relatively small air volume to the air channel when the second blower is in operation.

According to the above technical solutions, it can be found that the beneficial technical effects of the invention are: since the aromatherapy machine is provided with a plurality of internal air pressure configuration solutions, the atomized vapor can form a different mist retention state outside the aromatherapy machine. Therefore, a user can adjust different mist scenes by selecting different internal air pressure configuration solutions according to their surroundings and their moods. Therefore, the application of the aromatherapy machine can better coordinate with the surrounding environment and the user's own mood, thus improving the life experience and pleasure of the aromatherapy machine. Moreover, since the aromatherapy machine is provided with the first configuration solution and the second configuration solution, these two configuration solutions can produce two distinct mist scenes. The mist scene produced by the former forms a thick mountainous mist that slowly rises and is diffused slowly from the periphery of the mist storage cavity and drifted slowly downward, and the user can choose this configuration solution to reflect their peaceful mood. The mist scene produced by the latter imitates a natural scene of mist ejected high from a fountain, and the user can choose this configuration solution to reflect their happy mood. The two mist scenes can allow the user to obtain the joy of life, and also greatly increase the ornamental value of the aromatherapy machine.

### BRIEF DESCRIPTION OF THE DRAWINGS

FIG. 1 is a structural view of an aromatherapy machine 100 in a top view.

FIG. 2 is a diagram of the cross-sectional structure in an A-A direction in FIG. 1.

FIG. 3 is a diagram of the cross-sectional structure in a B-B direction in FIG. 1.

FIG. 4 is a three-dimensional structural diagram of a middle casing 12.

FIG. 5 is a cross-sectional view of another aromatherapy machine 100a applying the technical solution of the invention.

FIG. 6 is a diagram of the movement and change of the valve plate viewed along a direction of an arrow in FIG. 5.

FIG. 7 is a perspective structural diagram of an upper casing 11a.

### DESCRIPTION OF THE EMBODIMENTS

The concept, specific structure, and resulting technical effects of the invention are described clearly and completely in conjunction with the embodiments and drawings.

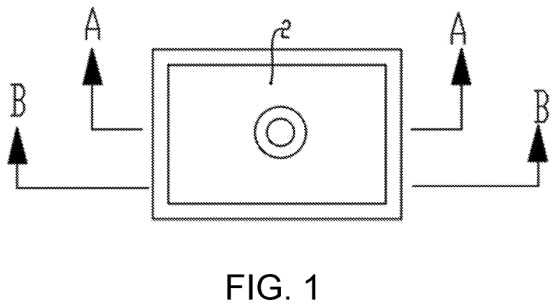

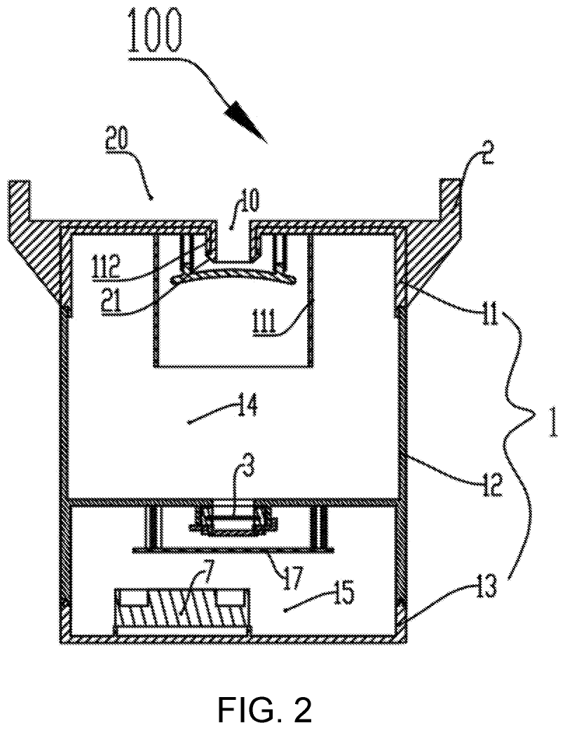

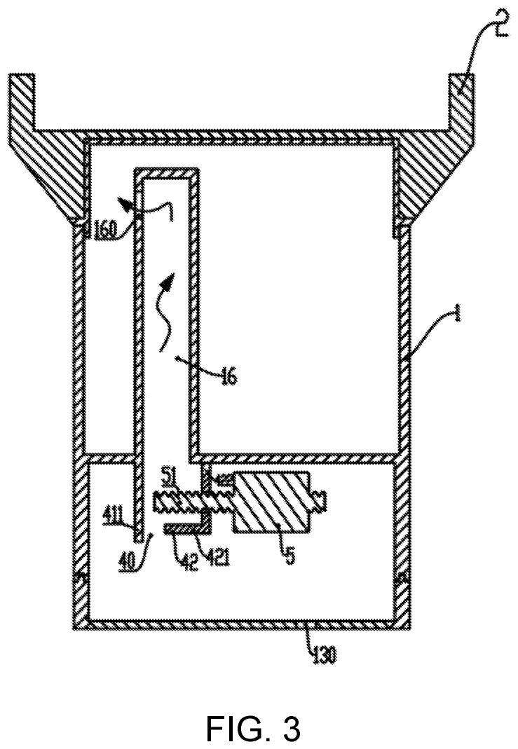

As shown in FIG. 1, FIG. 2, and FIG. 3, the invention provides a method for forming a changeable mist of an aromatherapy machine. A mist storage cavity 2 with an upward opening is disposed on the aromatherapy machine 100, and a mist outlet 10 of the aromatherapy machine 100 is communicated with an inner space 20 of the mist storage cavity 2. When the aromatherapy machine 100 generates an atomized vapor in an atomization chamber 14, the atomization chamber 14 is provided with an internal air pressure higher than an atmospheric pressure, so that the atomized vapor may be ejected outward from the mist outlet 10 at a corresponding different speed state based on a configured different internal air pressure to form a different mist retention state on an outside of the aromatherapy machine 100. A plurality of internal air pressure configuration solutions are provided, wherein in a first configuration solution, the internal air pressure of the atomization chamber 14 is controlled to be slightly higher than an external atmospheric pressure so that the atomized vapor floats out in a slow surge manner, so that the atomized vapor is mainly accumulated as a haze in the inner space 20 of the mist storage cavity 2. In a second configuration solution, the internal air pressure of the atomization chamber 14 is controlled to be higher than the internal air pressure controlled in the first configuration solution, so that the atomized vapor is ejected from the mist outlet 10 in a fast impulse manner, and that the atomized vapor is mainly scattered in a space above the mist storage cavity 2.

The structure of the aromatherapy machine 100 is first described in detail below. As shown in FIG. 1, FIG. 2, and FIG. 3, a body 1 of the aromatherapy machine 100 includes an upper casing 11, a base 13, and a middle casing 12 disposed between the upper casing 11 and the base 13. The atomization chamber 14 is formed between the upper casing 11 and the middle casing 12, and an electrical cavity 15 is formed between the middle casing 12 and the base 13. An ultrasonic atomizing sheet 3 configured to atomize an atomized liquid stored in the atomization chamber 14 is disposed on a bottom wall of the middle casing 12, and an electronic control board 17 that is signal-connected to the ultrasonic atomizing sheet 3 is disposed in the electrical cavity 15. A mist collecting tube 111 extended into the atomization chamber 14 is disposed at a top portion of the upper casing 11. The mist outlet 10 of the aromatherapy machine 100 is arranged with the mist exiting upward, and communicates with an inner space of the mist collecting tube 111 to also communicate with the atomization chamber 14. The mist outlet 10 is configured to discharge the atomized vapor in the atomization chamber 14 to a space outside the aromatherapy machine in an upward direction, and the droplets condensed in the mist storage cavity 2 can be reflowed via the mist outlet 10. An air channel 16 configured to provide an air supply amount to the atomization chamber 14 is further included. An air outlet 160 of the air channel 16 communicates with the atomization chamber 14. A blower 7 communicated with the air channel 16 is disposed in the electrical cavity 15.

The mist storage cavity 2 may adopt a structure integrally formed as one piece with a top portion of the aromatherapy machine 100. In the present embodiment, the mist storage cavity 2 is a member independent from the top portion of the aromatherapy machine 100. A connecting protrusion 112 extending into the atomization chamber 14 is disposed at the top portion of the upper casing 11. A buckle arm 21 is disposed at a bottom portion of the mist storage cavity 2. The mist storage cavity 2 is fastened to the connecting protrusion 112 via the buckle arm 21, so that the mist storage cavity 2 is detachably mounted on the top portion of the aromatherapy machine 100. The mist outlet 10 of the aromatherapy machine 100 communicates with the inner space 20 of the mist storage cavity 2. The mist storage cavity 2, the upper casing 11, and the middle casing 12 are light-transmitting bodies. A light-emitting body assembly (not shown in the figures) capable of changing colors is disposed below the mist storage cavity 2. The light-emitting body assembly is disposed on the electronic control board 17. The light-emitting body assembly is configured to set off the atomized vapor with the light it emits. In this way, the atomized vapor is colorful under the changing light storage capacity, and the viewing effect is better.

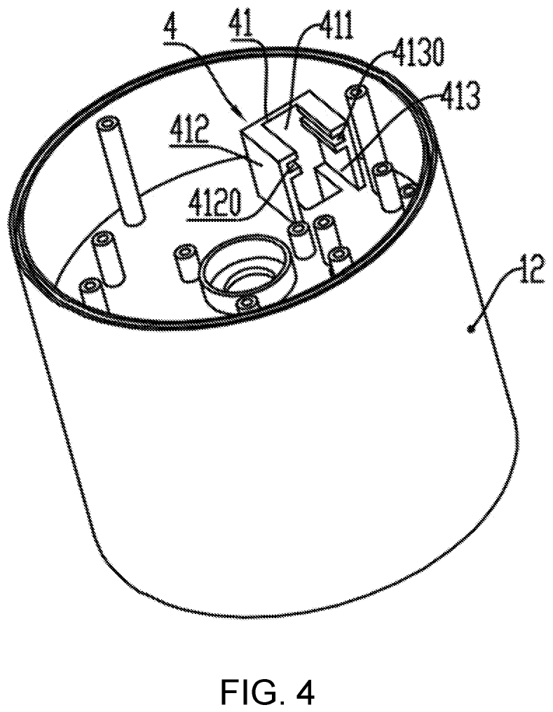

As shown in FIG. 3 and FIG. 4, an adjusting device is also included. The adjusting device is configured to adjust the internal air pressure of the atomization chamber 14 in a plurality of pressure configuration solutions. The atomized vapor can be ejected outward from the mist outlet 10 at a corresponding different speed state based on a different internal air pressure to form a different mist retention state on an outside of the aromatherapy machine 100. The adjusting device is an air valve 4 disposed on the air channel 16. The air valve 4 is configured to control the air supply amount provided via the air channel 16 to the atomization chamber 14 by changing the size of the air outlet. The air valve 4 includes a valve seat 41 and a valve plate 42 slidably disposed on the valve seat 41. The valve seat 41 includes left and right side walls (412, 413) and a back wall 411 disposed between the left and right side walls (412, 413). There are slide grooves (4120, 4130) are provided in the left and right side walls (412, 413) respectively. The valve plate 42 is L-shaped so as to include a lateral wall 421 and a vertical wall 422. The lateral wall 421 is slidably disposed on the slide grooves (4120, 4130). A gap between the lateral wall 421 and the back wall 411 is the air outlet 40 of the air valve 4.

A drive motor 5 mounted on the body of the aromatherapy machine 100 is also included. An output shaft 51 of the drive motor 5 is drivingly connected to the vertical wall 422 of the valve plate 42. The drive motor 5 is configured to drive the valve plate 42 to change the size of the air outlet 40 of the air valve 4 to control the air supply amount provided by the air channel 16 to the atomization chamber 14 to adjust the internal air pressure of the atomization chamber 14. When the drive motor 5 drives the valve plate 42 to retreat 5 mm from an original position to increase the size of the air outlet 40, the air channel 16 provides a relatively large air supply amount to the atomization chamber 14, so that the internal air pressure of the atomization chamber 14 is increased and is much higher than the atmospheric pressure. The atomized vapor in the atomization chamber 14 is ejected from the mist outlet 10 in a fast impulse manner. Most of the atomized vapor has a relatively high range and is scattered in the space above the mist storage cavity 2 to form a mist scene as if mist is ejected high from a fountain. A small part or no atomized vapor sinks into the mist storage cavity 2. When the drive motor 5 drives the valve plate 42 to advance 5 mm from the original position to reduce the size of the air outlet 40, the air channel 16 provides a relatively small air supply amount to the atomization chamber 14 so that the internal air pressure of the atomization chamber 14 drops to only slightly higher than atmospheric pressure. The atomized vapor floats out in a slow surge manner, so that the atomized vapor is mainly accumulated in the form of a haze in the inner space 20 of the mist storage cavity 2. It is as if a thick mountainous mist is formed that slowly rises and is diffused slowly from a periphery of the mist storage cavity 2 and slowly drifted downward, and as if a plume of fine smoke drifting from up high. The base 13 is also provided with a bottom hole 130, and excess air flow can take away the heat of the electronic control board 17 and is discharged from the bottom hole 130. It should be noted that the valve plate 42 is not limited to the above two position variations. Driven by the drive motor 5, the valve plate 42 can form a continuous position variation so that the internal air pressure of the atomization chamber 14 can be continuously adjusted. In this way, a plurality of internal air pressures can be formed, so as to form a plurality of mist scenes.

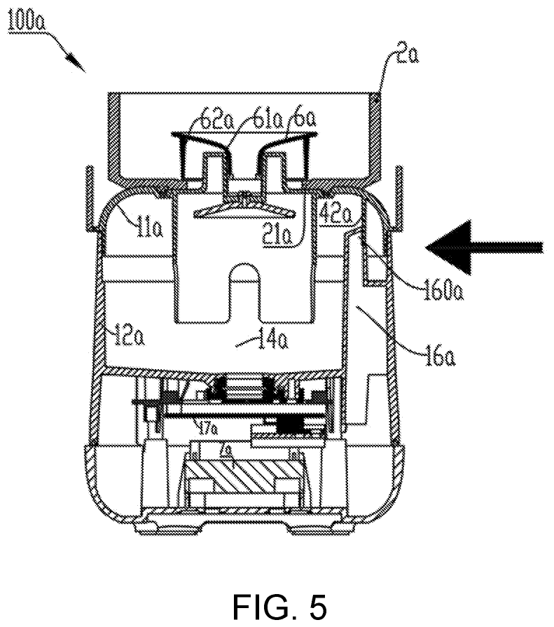

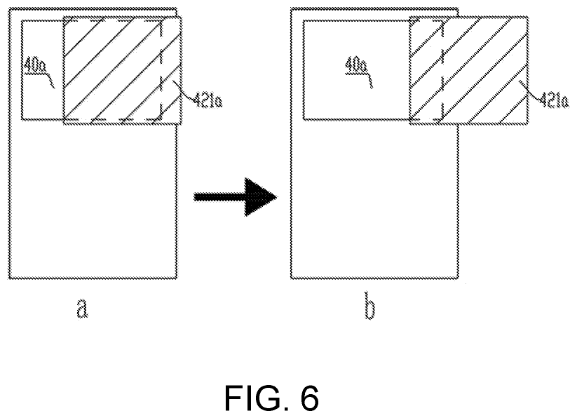

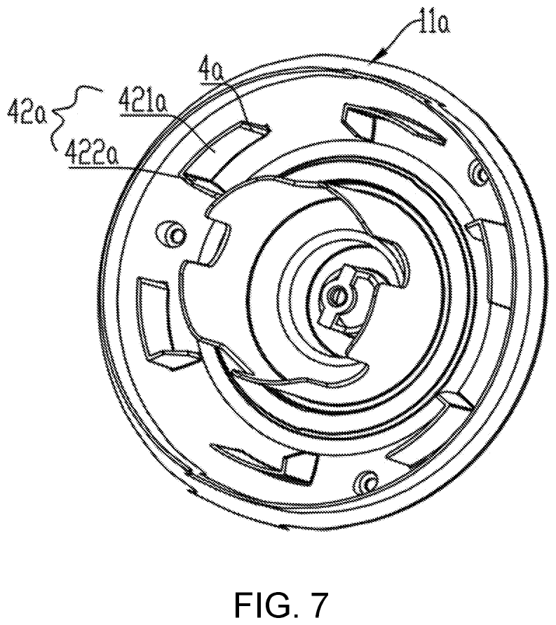

In addition to using the electric method to change the size of the air outlet of the air valve, a manual method can also be used. Another aromatherapy machine 100a is shown in FIG. 5, FIG. 6, and FIG. 7. The aromatherapy machine 100a has a similar structure to the aromatherapy machine 100 shown in FIG. 1 to FIG. 4, and the following focuses on the main differences between the two.

An upper casing 11a of the aromatherapy machine 100a is provided with a plurality of valve pieces 42a. The valve plate 42a is integrally formed as one piece with the upper casing 11a. The upper casing 11a is a valve seat that fixes the valve plate 42a. The valve plate 42a and the valve seat form an air valve 4a. The valve plate 42a is L-shaped so as to include a lateral wall 421a and a vertical wall 422a. The lateral wall 421a extends to the air outlet 160 of the air channel 16a to block the air outlet 160. The remaining overwind space is served as an overwind opening 40a of the air valve. A split structure is disposed between the upper casing 11a and the middle casing 12a, and the upper casing 11a is rotatable on the middle casing 12a to drive the valve plate 42a to rotate, so as to change the size of the overwind opening 40a. The vertical walls 422a of two adjacent valve plates 42a define the rotation range of the upper casing 11a relative to the air channel 16a. Since a plurality of the valve plates 42a are disposed, there is no strict restriction on the mounting direction of the upper casing 11a when the upper casing 11a is mounted on the middle casing 12a, thereby facilitating the mounting operation. In addition, the mist storage cavity 2a is also a member independent from a top portion of the aromatherapy machine 100a, and a bottom end of the mist storage cavity 2a is provided with a bent edge 21a. An auxiliary positioning member 6a is also included, wherein the auxiliary positioning member 6a includes a funnel-shaped main body 61a and a pressing edge 62a extending downward from the main body 61a. A funnel cavity of the auxiliary positioning member 6a communicates with an inner space 20a of the mist storage cavity 2 and an atomization chamber 14a. The main body 61a is fastened on the upper casing 11a. The bent edge 21a of the mist storage cavity 2a is pressed between the pressing edge 62a and the upper casing 11a.

Of course, it is possible to improve another aromatherapy machine 100a shown in FIG. 5, FIG. 6, and FIG. 7 by omitting the air valve 4a and adjusting the internal air pressure of the atomization chamber 14a by respectively changing the rotating speed of the blower 7a. For example, the adjusting device is an electronic control device (not shown in the figures) respectively mounted on the body of the aromatherapy machine 100a and can control the rotating speed of the blower 7a. The electronic control device is disposed on the electronic control board 17a. The electronic control device is configured to change the rotating speed of the blower 7a to control the air supply amount provided by the air channel 16a to the atomization chamber 14a. Alternatively, a dual fan solution is adopted, which is relatively simple and related figures are omitted. This solution includes a first blower and a second blower respectively communicating with the air channel. The air supply amount of the first blower is greater than the air supply amount of the second blower. The adjusting device is an electronic control device disposed on the body of the aromatherapy machine and capable of controlling the first blower and the second blower to be operated alternately. The electronic control device changes the operating state of the first blower or the second blower to control the air supply amount provided via the air channel to the atomization chamber. These two adjustment solutions can also be applied to the aromatherapy machine 100.

It can be known from the above technical solutions that, since the aromatherapy machine (100, 100a) is provided with a plurality of internal air pressure configuration solutions, the atomized vapor can form a different mist retention state outside the aromatherapy machine (100, 100a). Therefore, users can adjust different mist scenes by selecting different internal air pressure configuration solutions according to the surrounding environment and their own mood. Therefore, the application of the aromatherapy machine (100, 100a) can better coordinate with the surrounding environment and the user's own mood, thus improving the life experience of the aromatherapy machine. And because the aromatherapy machine (100, 100a) is provided with the first configuration solution and the second configuration solution, these two configuration solutions can produce two completely different mist scenes. The mist scene produced by the former forms a natural scene that imitates a mountainous thick mist that slowly rises and is diffused from the periphery of the mist storage cavity and slowly drifted downward. Users can choose this configuration solution to reflect their peace of mind. The mist scene produced by the latter imitates a natural scene of mist ejected high from a fountain. Users can choose this configuration solution to reflect their happy mood. The two mist scenes provide users with a sense of the joy of life, and also greatly increase the ornamental value of the aromatherapy machine (100, 100a).

1. A method for forming a changeable mist of an aromatherapy machine, the method comprising:
providing a mist storage cavity with an upward opening on the aromatherapy machine, wherein a mist outlet of the aromatherapy machine is communicates with an inner space of the mist storage cavity;
when the aromatherapy machine generates an atomized vapor in an atomization chamber, providing the atomization chamber with an internal air pressure higher than an atmospheric pressure, so that the atomized vapor is ejected outward from the mist outlet at a corresponding different speed state based on a different internal air pressure to form a different mist retention state on an outside of the aromatherapy machine; and
providing a plurality of internal air pressure configuration solutions, wherein in a first configuration solution, the internal air pressure of the atomization chamber is controlled to be slightly higher than an external atmospheric pressure, so that the atomized vapor floats out in a slow surge manner, and that the atomized vapor is mainly accumulated as a haze in the inner space of the mist storage cavity, and in a second configuration solution, the internal air pressure of the atomization chamber is controlled to be higher than the internal air pressure controlled in the first configuration solution, so that the atomized vapor is ejected from the mist outlet in a fast impulse manner, and that the atomized vapor is mainly scattered in a space above the mist storage cavity.

2. The method for forming the changeable mist of claim 1, wherein the mist storage cavity is a light-transmitting body, and a changing light is disposed below the mist storage cavity to set off the atomized vapor.

3. The method for forming the changeable mist of claim 1, wherein the internal air pressure of the atomization chamber is continuously and variably controllable.

4. The method for forming the changeable mist of claim 1, further comprising disposing an air channel providing an air supply amount to the atomization chamber on the aromatherapy machine, and controlling the air supply amount provided to the atomization chamber via the air channel to configure the internal air pressure of the atomization chamber.

5. The method for forming the changeable mist of claim 4, wherein a size of an air outlet is changed via an air valve disposed on the air channel, so as to control the air supply amount provided to the atomization chamber via the air channel.

6. The method for forming the changeable mist of claim 4, wherein the air supply amount provided to the atomization chamber via the air channel is controlled by controlling a rotating speed of a blower communicating with the air channel.

7. An aromatherapy machine implementing a method for forming a changeable mist of the aromatherapy machine, the aromatherapy machine comprising an atomization chamber disposed in a body of the aromatherapy machine, and a mist outlet arranged in an upward direction, wherein the mist outlet communicating with the atomization chamber to discharge an atomized vapor in the atomization chamber to a space outside the aromatherapy machine in the upward direction; a mist storage cavity with an opening upward is disposed on the aromatherapy machine, and the mist outlet of the aromatherapy machine communicates with an inner space of the mist storage cavity; the aromatherapy machine further comprising an adjusting device, wherein the adjusting device is configured to adjust an internal air pressure of the atomization chamber in a plurality of pressure configuration solutions, so that the atomized vapor is ejected outward from the mist outlet at a corresponding different speed state based on a different internal air pressure to form a different mist retention state on an outside of the aromatherapy machine; in a first configuration solution, the adjusting device is configured to control the internal air pressure of the atomization chamber to be slightly higher than an external atmospheric pressure, so that the atomized vapor floats out in a slow surge manner, and that the atomized vapor is mainly accumulated as a haze in the inner space of the mist storage cavity, and in a second configuration solution, the adjusting device is configured to control the internal air pressure of the atomization chamber to be higher than the internal air pressure controlled in the first configuration solution, so that the atomized vapor is ejected from the mist outlet in a fast impulse manner, and that the atomized vapor is mainly scattered in a space above the mist storage cavity.

8. The aromatherapy machine of claim 7, wherein the mist storage cavity is a light-transmitting body, a light-emitting body assembly that has changeable colors is disposed below the mist storage cavity, and the light-emitting body assembly is configured to set off the atomized vapor with a light emitted thereby.

9. The aromatherapy machine of claim 7, wherein the mist storage cavity is integrally formed as one piece with a top portion of the aromatherapy machine, or the mist storage cavity is independent from the top portion of the aromatherapy machine and the mist storage cavity is detachably installed on the top portion of the aromatherapy machine.

10. The aromatherapy machine of claim 7, further comprising an air channel communicating with the atomization chamber, wherein the air channel is configured to provide an air supply amount to the atomization chamber; the adjusting device is configured to control the air supply amount provided to the atomization chamber via the air channel to adjust the internal air pressure of the atomization chamber.

11. The aromatherapy machine of claim 10, wherein the adjusting device is an air valve disposed on the air channel, and the air valve is configured to control the air supply amount provided to the atomization chamber via the air channel by changing a size of the air outlet.

12. The aromatherapy machine of claim 11, further comprising a drive motor mounted on a body of the aromatherapy machine, wherein an output shaft of the drive motor is drivingly connected to the air valve, and the drive motor is configured to drive an action of the air valve to change the size of the air outlet of the air valve.

13. The aromatherapy machine of claim 10, further comprising a blower communicating with the air channel, wherein the adjusting device is an electronic control device disposed on the body of the aromatherapy machine to control a rotating speed of the blower, and the electronic control device is configured to change the rotating speed of the blower to control the air supply amount provided to the atomization chamber via the air channel.

14. The aromatherapy machine of claim 10, further comprising a first blower and a second blower respectively communicating with the air channel, wherein an air supply amount of the first blower is greater than an air supply amount of the second blower, the adjusting device is an electronic control device disposed on the body of the aromatherapy machine to control the first blower and the second blower to be operated alternately, and an operating state of the first blower or the second blower is changed via the electronic control device to control the air supply amount provided to the atomization chamber via the air channel.

15. The method for forming the changeable mist of claim 3, further comprising disposing an air channel providing an air supply amount to the atomization chamber on the aromatherapy machine, and controlling the air supply amount provided to the atomization chamber via the air channel to configure the internal air pressure of the atomization chamber.
